(19) United States
(12) Patent Application Publication
(10) Pub. No.: US 2026/0069805 A1
(43) Pub. Date: Mar. 12, 2026

(54) METHOD AND SYSTEM FOR AUTOMATING OXYGEN MONITORING AND DOSING IN REAL TIME FOR PATIENT ON OXYGEN THERAPY

(71) Applicant: VANDERBILT UNIVERSITY, Nashville, TN (US)

(72) Inventor: Lisa Hood Lancaster, Nashville, TN (US)

(21) Appl. No.: 19/394,225

(22) Filed: Nov. 19, 2025

Related U.S. Application Data
(63) Continuation-in-part of application No. 17/492,822, filed on Oct. 4, 2021, now abandoned.
(60) Provisional application No. 63/086,957, filed on Oct. 2, 2020.

(51) Int. Cl. A61M 16/00; A61B 5/00; A61B 5/01; A61B 5/0205; A61B 5/021; A61B 5/024; A61B 5/08; A61B 5/11; A61B 5/1455

(52) U.S. Cl. CPC A61M 16/026; A61B 5/0004; A61B 5/01; A61B 5/0205; A61B 5/021; A61B 5/024; A61B 5/0816; A61B 5/1126; A61B 5/14552; A61B 5/4836; A61B 5/6824; A61B 5/6829; A61B 5/7264; A61B 5/7271; A61B 5/742; A61B 5/746; A61B 5/747; A61M 16/0003; A61M 16/0051; A61B 2560/0462; A61B 2562/0219; A61B 2562/0271; A61M 2202/0007; A61M 2202/0208; A61M 2205/18; A61M 2205/3303; A61M 2205/3306; A61M 2205/3327; A61M 2205/3334; A61M 2205/3344; A61M 2205/3368; A61M 2205/3561; A61M 2205/3584; A61M 2205/502; A61M 2205/583; A61M 2209/088; A61M 2210/083; A61M 2210/086; A61M 2230/005; A61M 2230/04; A61M 2230/20; A61M 2230/42; A61M 2230/50; A61M 2230/63

(57) ABSTRACT

This system and method for automating oxygen monitoring and dosing in real time for a patient on oxygen therapy is disclosed. The system includes a wearable sensor device configured to measure oxygen saturation levels of the patient; track physical activity of the patient; and transmit real-time oxygen saturation and activity data; and a smart flow regulator operably coupled to an oxygen source and configured to receive the transmitted data from the wearable sensor device; implement an adaptive control algorithm to determine an appropriate oxygen flow rate based on the received oxygen saturation and activity data; and automatically adjust a flow rate of oxygen delivered from the oxygen source to the patient in real time.

### CROSS-REFERENCE TO RELATED PATENT APPLICATIONS

This application is a continuation in part application of U.S. patent application Ser. No. 17/492,822, filed Oct. 4, 2021, which itself claims priority to and the benefit of U.S. Provisional Patent Application Ser. No. 63/086,957, filed Oct. 2, 2020, which are incorporated herein in their entireties by reference.

### FIELD OF THE INVENTION

The present invention relates generally to medical devices and patient monitoring systems, and more particularly to a system and method for automating oxygen monitoring and dosing in real time for a patient receiving supplemental oxygen therapy. The invention combines wearable sensing, smart flow regulation, and adaptive control algorithms to dynamically adjust oxygen delivery based on patient activity and physiological status.

### BACKGROUND OF THE INVENTION

The background description provided herein is for the purpose of generally presenting the context of the invention. The subject matter discussed in the background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

It is estimated that up to 1.5 million people in the United States use supplemental oxygen, but confident numbers are lacking. As more patients are living longer and being diagnosed with lung diseases, the numbers are anticipated to continue to increase. Common causes of hypoxic lung disease include chronic obstructive pulmonary disease (COPD), emphysema, interstitial and fibrotic lung diseases, cardiac disease such as congestive heart failure and ischemic heart disease as well as infectious or inflammatory lung diseases. Oxygen (O₂) therapy has become standard of care for support of hypoxic lung disease patients and an integral component of care for patients with idiopathic pulmonary fibrosis. Oxygen therapy has been shown in certain pulmonary diseases to improve endurance, walking distance, shortness of breath, improve sleep, improve cognitive function, improve survival, and prevent pulmonary hypertension with subsequent right heart failure.

Despite the large numbers of patients needing both home and portable oxygen, little progress has been made in the development of oxygen devices in the last few decades. Patients remain burdened with cumbersome, heavy tanks, long tubing, frequent need to change tanks if high oxygen flow rates are needed, and the inability to easily change the flow rate on stationary tanks if moving around the home. This can worsen with progressive disease requiring higher flow rates and more oxygen.

Presently, home oxygen prescriptions are written based on the provider's assessment of oxygen needs and prescribed a certain fixed liter flow rate starting at 2 liters per minute. Providers will estimate the needs based on resting oxygen saturations and oxygen saturations during walks in clinic or the hospital while wearing a pulse oximeter. Oxygen is then adjusted or titrated to keep the pulse oximeter saturations in the 90s while walking and at rest. The flow rate of oxygen needed to keep O₂ saturations in the 90s at rest and with walking is then prescribed at a fixed rate for the patient to self-adjust between resting and exertion or sleep. As patients become more active in their home lifting, carrying and bending, their oxygen needs increase beyond walking on a flat surface setting up a scenario for further hypoxia while on oxygen in the home. Estimates in clinic walking on a flat surface do not predict home needs well.

This becomes logistically problematic in the home due to the reality of patients needing more oxygen when they are more active than when they are at rest. The oxygen concentrator for patients is set up in one room in the house which is usually the bedroom. A patient will walk from the bedroom to the living room or another room in the house and will need a higher flow rate when walking or exerting themselves than when they are at rest. Now they are in the living room planning to sit in a chair so oxygen by prescription should be turned down at rest but they are in the living room far from the oxygen concentrator and regulator that adjusts the flow rate of the oxygen. This continues throughout the day as they move from sitting to room to room. The fixed prescribed rates of oxygen flow for rest and walking do not capture the needs when the patient may be exerting themselves more with other daily activities.

Most patients either over utilize oxygen and place it on the flow rate needed with exertion or under dose the oxygen leaving it on the flow rate needed at rest or somewhere in between. All may be problematic. Higher dosing could develop pro inflammatory free radicals aggravating underlying lung disease. Under dosing oxygen puts patients at risk of pulmonary hypertension and right heart failure, lower endurance, risk of arrythmia, risk of syncope and risk of ischemia. Having the ability to adjust flow rate in real time with the rise and fall of oxygen saturations during rest and with activity would be even more advantageous with portable oxygen. Being able to adjust down the oxygen with sitting as oxygen saturations allow, would enable the patient to conserve the oxygen in the tank resulting in fewer tanks being needed to travel outside the home.

In addition, patients on oxygen in hospitals are adjusted by the nurse who may notice alarms for low oxygen levels or tachycardia and respond by leaving one patient's room and moving to the one with alarms sounding. Due to location and the duties the nurse is performing with another patient, a time delay in adjustment of oxygen occurs. A real time device that sounds the alarm while adjusting the oxygen per software protocol to the flow rate needed by the patient to maintain normoxia would correct hypoxia sooner while avoiding lost time and the numerous complications of hypoxia most notably arrythmia with cardiopulmonary arrest in the acutely ill patient.

Therefore, a heretofore unaddressed need exists in the art to address the aforementioned deficiencies and inadequacies.

### SUMMARY OF THE INVENTION

One of the objectives of this invention is to provide a real-time, closed-loop oxygen monitoring and dosing system comprising a wearable sensor device, a smart flow regulator, and a controller executing an adaptive titration algorithm, to overcome the limitations of conventional systems.

The wearable sensor continuously measures physiological parameters including oxygen saturation (SpO₂) and physical activity via integrated sensors such as a pulse oximeter and accelerometer. These data are wirelessly transmitted to a controller, such as a smartphone or tablet, executing an application that analyzes incoming data and determines the appropriate oxygen flow rate. The controller communicates with a smart flow regulator—mechanically or electronically coupled to a standard oxygen concentrator or tank—to adjust the oxygen flow rate accordingly in real time.

The smart flow regulator includes a stepper motor mechanically linked to a flow adjustment knob or electronically linked to a digital concentrator, and a driver circuit that operably interprets control signals from the controller and actuates the motor to change flow rate. Manual override capability is provided for safety in case of communication failure.

In case of communication interruption, the smart flow regulator halts at the last known safe setting and allows manual control. The controller application displays oxygen saturation, flow rate, and activity level, and provides alerts for low SpO₂, sensor disconnection, or high pulse rate.

The algorithm incorporates proportional-integral-derivative (PID) logic and time-averaged oxygenation analysis (10-20 seconds) to account for physiologic delay in oxygen uptake and avoid overcorrection. It further recognizes rest versus activity states from actigraphy data to apply baseline flow rate profiles pre-set by a healthcare provider.

All data are transmitted wirelessly among the wearable device, the controller, and the flow regulator. The controller can store data locally or upload it securely to a cloud database for provider review. The application can generate daily or weekly summaries of oxygen saturation, flow rate adjustments, and activity patterns.

If oxygen saturation falls below a critical threshold, the system triggers alarms, instructs the patient to rest, and can automatically contact emergency services or alert caregivers. The system also stores oxygenation, activity, and flow data locally or on a secure cloud platform for provider access and long-term tracking.

Novelty and advantages of the invention include, but are not limited to: fully automated, closed-loop system: integrates measurement, analysis, and actuation without manual intervention; smart flow regulator: motorized or digital control capable of interfacing with existing concentrators/tanks; adaptive algorithm: accounts for physiologic delay, activity level, and individual variability; safety features: multi-level alarms, fail-safe override, and emergency call initiation; and clinical and economic benefits: improved oxygenation accuracy, reduced oxygen waste, and enhanced patient independence.

The invention thus represents a significant advancement in respiratory care by enabling continuous, adaptive oxygen therapy tailored to each patient's real-time needs.

In one aspect of the invention, the system for automating oxygen monitoring and dosing in real time for a patient on oxygen therapy comprises a wearable sensor device configured to measure oxygen saturation levels of the patient; track physical activity of the patient; and transmit real-time oxygen saturation and activity data; and a smart flow regulator operably coupled to an oxygen source and configured to receive the transmitted data from the wearable sensor device; implement an adaptive control algorithm to determine an appropriate oxygen flow rate based on the received oxygen saturation and activity data; and automatically adjust a flow rate of oxygen delivered from the oxygen source to the patient in real time.

In one embodiment, the wearable sensor device comprises at least one pulse oximeter and at least one accelerometer integrated within a wearable housing configured to be worn on a wrist, arm, ankle, or other body part of the patient.

In one embodiment, the system further comprises a controller configured to coordinate data exchange, execute the adaptive control algorithm, and maintain the oxygen saturation level within a predetermined target range.

In one embodiment, the smart flow regulator comprises a stepper motor mechanically coupled to a flow adjustment member of the oxygen source; and a driving circuit configured to receive control signals from the controller and drive the stepper motor to adjust the oxygen flow rate accordingly.

In one embodiment, the smart flow regulator is configured for digital or mechanical coupling with a home or portable oxygen concentrator or tank.

In one embodiment, the smart flow regulator includes an emergency override mechanism permitting manual flow rate adjustment in the event of communication failure.

In one embodiment, the adaptive control algorithm comprises a proportional-integral-derivative (PID) control logic configured to smooth variations in oxygen saturation and prevent overcorrection.

In one embodiment, the controller operably averages measured oxygen saturation data over a predetermined interval of 10-20 seconds prior to calculating flow rate adjustments.

In one embodiment, the controller is configured to classify the physical activity level of the patient into resting and active states based on actigraphy data, and to apply corresponding baseline oxygen flow rates for each state.

In one embodiment, the baseline oxygen flow rates for resting and active states are pre-configured by a healthcare provider.

In one embodiment, the controller operably executes a titration protocol comprises maintaining the current flow rate when oxygen saturation is between 90% and 95%; increasing the flow rate when oxygen saturation falls below 90%; and reducing the flow rate when oxygen saturation exceeds 95%.

In one embodiment, the controller is further configured to trigger an alarm or visual notification when oxygen saturation falls below a predetermined threshold; and prompt the patient to rest or automatically contact emergency services if the low oxygen condition persists for a set time period.

In one embodiment, the controller is configured to generate daily or weekly reports of oxygen saturation, activity level, and oxygen flow data accessible to a healthcare provider via a secure portal.

In one embodiment, communication between the wearable sensor device, the controller, and the smart flow regulator is achieved wirelessly using at least one of Bluetooth®, Wi-Fi, or cellular communication protocols.

In one embodiment, the controller is integrated in the smart flow regulator, the wearable sensor device, or an external device.

In one embodiment, the external device comprises a smartphone or tablet executing a software application configured to receive oxygen saturation and activity data from the wearable sensor device; transmit flow control signals to the smart flow regulator; display real-time physiological and system information; and store data locally or on a remote server for review by healthcare providers.

In one embodiment, the wearable sensor device is further configured to monitor additional physiological parameters including heart rate, respiratory rate, skin temperature, and blood pressure, and wherein the controller adjusts oxygen delivery based on one or more of said parameters.

In another aspect of the invention, the method for automating oxygen monitoring and dosing in real time for a patient on oxygen therapy comprises measuring oxygen saturation levels of the patient with a wearable sensor device; tracking physical activity of the patient with the wearable sensor device; transmitting real-time oxygen saturation and activity data from the wearable sensor device to a controller; processing the transmitted data using an adaptive control algorithm executed by the controller to determine an oxygen flow rate appropriate for the patient's physiological and activity state; and automatically adjusting a smart flow regulator operably coupled to an oxygen source to deliver oxygen at the determined flow rate.

In one embodiment, the method further comprises classifying the activity level of the patient as resting or activity based on actigraphy data, and applying corresponding baseline oxygen flow rates for each state.

In one embodiment, the adaptive control algorithm comprises a proportional-integral-derivative (PID) feedback system configured to incrementally adjust oxygen flow based on averaged oxygen saturation readings over a time window of 10-20 seconds.

In one embodiment, the method further comprises maintaining the oxygen saturation level within a target range of 90% to 95% by increasing the flow rate when oxygen saturation falls below the target range; and decreasing the flow rate when oxygen saturation exceeds the target range.

In one embodiment, the method further comprises triggering an alarm and notifying the patient to rest or seek assistance when the oxygen saturation level falls below a critical threshold for a predetermined time period.

In one embodiment, the method further comprises storing oxygen saturation, activity, and flow rate data locally or transmitting the data to a cloud database accessible to a healthcare provider.

In one embodiment, the smart flow regulator comprises a stepper motor operably connected to a mechanical or digital flow control member of an oxygen concentrator or tank.

In one embodiment, the method further comprises averaging oxygen saturation readings over a predetermined interval to account for physiologic delay in oxygen uptake prior to adjusting the flow rate.

In one embodiment, the method further comprises automatically initiating an emergency call or provider alert if oxygen saturation remains below a critical level for a predetermined duration.

In one embodiment, communication between the wearable sensor device, the controller, and the smart flow regulator is performed via a wireless communication protocol selected from the group consisting of Bluetooth®, Wi-Fi, and cellular communication.

In one embodiment, the method further comprises generating and displaying in a software application a graphical summary of oxygen saturation trends, activity levels, and corresponding oxygen flow rates for review by a healthcare provider.

In one embodiment, the adaptive control algorithm further utilizes historical patient data to predict oxygen needs and pre-emptively adjust the flow rate during anticipated periods of exertion.

In yet another aspect of the invention, the method for personalized oxygen delivery to a user on oxygen therapy comprises continuously monitoring an oxygen saturation level of the user with a wearable oxygen sensor; tracking, in real time, a physical activity level of the user using an activity sensor integrated with or communicatively coupled to the wearable oxygen sensor; dynamically adjusting a flow rate of oxygen delivered from an oxygen source to the user in response to at least one of the detected physical activity level; and the measured oxygen saturation level; incrementally increasing or decreasing the oxygen flow rate at predetermined intervals in accordance with an adaptive titration protocol, thereby maintaining the oxygen saturation level within a predetermined range; and reassessing the oxygen saturation data after each incremental flow rate adjustment to determine whether further adjustment is required.

In one embodiment, the reassessment of oxygen saturation occurs approximately every two minutes following each flow rate adjustment.

In one embodiment, the step of incrementally adjusting the oxygen flow rate comprises increasing the flow rate by predetermined increments when the oxygen saturation falls below a threshold level and decreasing the flow rate when the oxygen saturation exceeds an upper limit.

In one embodiment, the method further comprises classifying the user's activity level as one of a resting state or an active state, and initiating adjustment of the flow rate from respective baseline oxygen flow rates associated with the classified state.

In one embodiment, the dynamically adjusting step is performed automatically by a controller executing an adaptive algorithm configured to interpret both oxygen saturation and activity data.

In one embodiment, the adaptive algorithm comprises a proportional-integral-derivative (PID) feedback control that smooths variations in oxygen saturation and compensates for physiological delay in oxygen uptake.

In one embodiment, the method further comprises averaging measured oxygen saturation values over a predetermined time window of about 10-20 seconds prior to each reassessment.

In one embodiment, the method further comprises triggering an alert when the oxygen saturation falls below a predetermined critical threshold for longer than a preset duration.

In one embodiment, the incremental adjustment of the oxygen flow rate occurs in discrete steps of approximately 1 liter per minute (L/min) or less.

In one embodiment, the predetermined reassessment interval and adjustment increments are configurable by a healthcare provider via a software interface.

In one embodiment, the method further comprises storing oxygen saturation, activity level, and flow rate data in a local or cloud-based memory for review by a healthcare provider.

In one embodiment, the dynamically adjusting step is based solely on detected physical activity when valid oxygen saturation data are unavailable or outside a confidence threshold.

In one embodiment, the reassessment interval and incremental flow changes are coordinated such that no flow adjustment is performed until a stable oxygen saturation value has been confirmed over a preceding assessment window.

In one embodiment, the method further comprises predicting upcoming oxygen demands based on historical activity data of the user and preemptively adjusting the oxygen flow rate to maintain desired oxygen saturation levels.

In one embodiment, the method further comprises displaying, on a graphical user interface, real-time oxygen saturation, activity level, and current flow rate of oxygen delivery.

These and other aspects of the invention will become apparent from the following description of the preferred embodiments, taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. The same reference numbers may be used throughout the drawings to refer to the same or like elements in the embodiments.

FIG. 1 shows schematically a system for automating oxygen monitoring and dosing in real time for a patient on oxygen therapy according to one embodiment of the invention.

FIG. 2 shows schematically a diagram of an application protocol for automating oxygen monitoring and dosing in real time for a patient on oxygen therapy according to one embodiment of the invention.

FIG. 3 shows schematically a system for automating oxygen monitoring and dosing in real time for a patient on oxygen therapy according to another embodiment of the invention.

FIG. 4 shows schematically a circuit diagram for a flow regulator according to one embodiment of the invention.

FIG. 5 shows schematically a connection member for a flow regulator according to one embodiment of the invention.

FIG. 6 shows schematically a cover box for a flow regulator according to one embodiment of the invention.

FIGS. 7A-7D show schematically pages of a customized application (AutO2 app) for automating oxygen monitoring and dosing in real time for a patient on oxygen therapy according to one embodiment of the invention.

FIG. 8 shows schematically a display of the AutO2 app according to one embodiment of the invention.

FIG. 9 shows a rate of oxygen delivery according to one embodiment of the invention.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

It will be understood that, as used in the description herein and throughout the claims that follow, the meaning of “a”, “an”, and “the” includes plural reference unless the context clearly dictates otherwise. Also, it will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending of the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

It will be further understood that the terms “comprises” and/or “comprising,” or “includes” and/or “including” or “has” and/or “having”, or “carry” and/or “carrying,” or “contain” and/or “containing,” or “involve” and/or “involving, and the like are to be open-ended, i.e., to mean including but not limited to. When used in this invention, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used herein, the phrase at least one of A, B, and C should be construed to mean a logical (A or B or C), using a non-exclusive logical OR. It should be understood that one or more steps within a method may be executed in different order (or concurrently) without altering the principles of the present disclosure.

The systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more example embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

One of the objectives of this invention is to provide systems and methods for oxygen monitoring with flow rate adjustment of oxygen in real time. One exemplary embodiment of the system includes a bracelet like device that captures pulse oximetry oxygen saturation data and activities in real time and is linked to both an app complete with software providing an oxygen titration protocol that is then linked with the flow rate regulator. This system is wirelessly coupled with home concentrators and portable oxygen tanks to seamlessly provide information and instruction to the regulator to adjust the flow rate to the oxygen needs of the patient. In one embodiment, this system is adapted for automating oxygen monitoring and dosing in real time for a patient on home or portable oxygen therapy. The system includes a flow regulator coupled to an oxygen source for operably controlling a flow rate of oxygen from the oxygen source to the patient; a wearable device configured to be worn by the patient for measuring physiological parameters of oxygen saturation among others; and a controller application in wireless communication with the wearable device and the flow regulator. After receiving the oxygen saturation data, the controlling operations of the application on the smart phone regulates the flow rate of oxygen from the oxygen source for providing a desired amount of oxygen to the patient based on the physiological status of the patient. This is the first device of its kind to treat hypoxia in real time and allow for conservation of oxygen without risk of under dosing the patient.

The novel device allows for conservation of a medication, appropriate dosing of that medication and safety in medication dispensing and dose adjustment. That medication is oxygen which is a key treatment for patients with idiopathic pulmonary fibrosis (IPF) or other diseases with hypoxic respiratory failure. A significant advantage to IPF patients and others with hypoxic lung disease would be the peace of mind, if a device like this would provide allowing patients to have their oxygen appropriately dosed without having to think of adjusting the flow rate numerous times during the course of the date. As a patient, going about your regular daily life mentally and physically unencumbered by no longer constantly adjusting the oxygen dial should contribute significantly to quality of life improvements. With the limitations of oxygen delivery by durable medical equipment companies, limitations in portability of oxygen and growing limitations in medical oxygen supply due to CoVID-19 related lung disease in some regions of the world, a device such as this could be invaluable for care of the acutely and chronically hypoxic patient.

This solution utilizes and marries existing technology (a wearable SpO₂ monitor, cloud based data storage) with a unique algorithm that processes the SpO₂ data and directs a novel mechanical or digital smart flow knob to adjust the oxygen flow rate accordingly. This combination of technology and algorithm coupled with data storage of SpO₂ information and needed or utilized flow rates is not present in publicly available reports or papers and certainly not utilized in the real world. Prior devices in the area have not developed appropriate algorithms that successfully supplement oxygen to keep oxygen saturations in the 90 sec for any clinically meaningful length of time. They lack consideration of concepts of physiologic delay for uptake of oxygen, circulation, and time to display of the oxygen saturation data. No remote control oxygen delivery device is presently in the marketplace for patients.

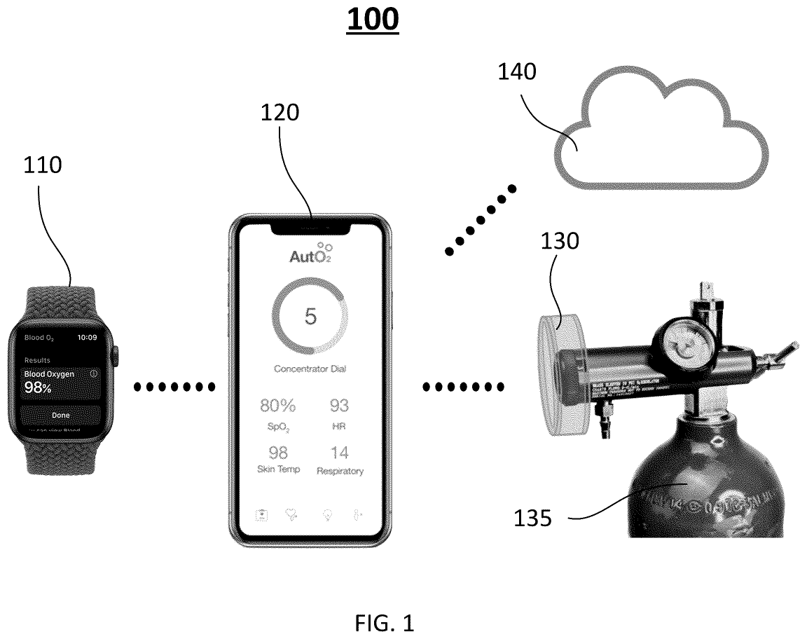

Referring to FIG. 1, a system 100 for automating oxygen monitoring and dosing in real time for a patient on oxygen therapy is shown according to one embodiment of the invention. When a patient is on oxygen therapy, the patient is administrated with an amount/dose of oxygen usually by a tube connecting between the patient and an oxygen source such as an oxygen tank. The amount/dose of oxygen should be adjustable based on patent's physiological need or status in real time. The physiological need or status can be evaluated or determined by patient's physiological parameters in real time, such as, but are not limited to, a SpO₂, a heart rate, a respiratory rate, a temperature, activity level, and/or a blood pressure.

In the exemplary embodiment shown in FIG. 1, the system 100 includes a flow regulator 130 coupled to an oxygen source 135 for operably controlling a flow rate of oxygen from the oxygen source 135 to the patient; a wearable device 110 configured to be worn by the patient for measuring physiological parameters of the patient to monitor a physiological status of the patient; and a controller 120 in wireless communication with the wearable device 110 and the flow regulator 130 for receiving the measured physiological parameters from the wearable device 110, and controlling operations of the flow regulator 130 to regulate the flow rate of oxygen from the oxygen source 135 for providing a desired amount of oxygen to the patient based on the physiological status of the patient. The wireless communication is performed via the internet, WiFi, API, and/or Bluetooth protocols.

The flow regulator 130 is a mechanical or digital smart flow knob that is operably engaged with a standard oxygen concentrator/tank 135 and is capable of automatically adjusting the oxygen flow rate from the oxygen tank 135 to the patient according to the patient's physiological status that is determined by the measured physiological parameters. In some embodiments, the flow regulator 130 comprises a stepper motor; a connection member engaged with the stepper motor and the oxygen source 135 such that rotation of the stepper motor drives the connection member to rotate, which in turn adjusts the flow rate of oxygen from the oxygen source 135; and a driving circuit in communication with the controller 120 and the stepper motor for receiving the signal from the controller 120 and driving the stepper motor with the signal so as to adjust the flow rate of oxygen accordingly. In one embodiment, the driving circuit includes an API/Bluetooth module/circuit/driver for the wireless communication with the controller 120, and/or the wearable device 110.

In other embodiments, some portable oxygen concentrators and home oxygen concentrators are digital and electronically driven instead of driven or adjusted by a rotary knob. For the digital concentrators, accessibility for adjustment is included in this concept. For example, the flow regulator is configured such that the flow rate of the digital concentrators is electronically driven through digital means. In other embodiments, the flow regulator comprises digital means for adjusting the flow rate of the digital concentrators electronically.

The wearable device 110 is essentially a sensor device that includes one or more pulse oximeters, and/or one or more pulse co-oximeter for measuring the SpO₂ of the patient.

In some embodiments, the wearable device 110 may also include at least one accelerometer for motion measurements for evaluating a rest or active (e.g., walking) state of the patient; and/or at least one temperature sensor for temperature measurements.

In some embodiments, the wearable device 110 is further configured to be in wireless communication with the flow regulator 130.

In some embodiments, the wearable device 110 is configured to be worn by the patient on an ankle, a wrist, a calf, a thigh, an upper arm, or other applicable body part for oxygen saturation detection.

In one embodiment, the wearable device 110 may also include an API/Bluetooth module/circuit/driver for the wireless communication with the controller 120, and/or the flow regulator 130.

The controller 120 is wirelessly coupled with the wearable device 110 and the flow regulator 130. It is adapted for receiving the measured physiological parameters from the wearable device 110 and controlling operations of the flow regulator 130 to regulate the flow rate of oxygen from the oxygen source 135 for providing a desired amount of oxygen to the patient based on the physiological status of the patient. Specifically, when the physiological parameter, e.g., the SpO₂, is received from the wearable sensor 110, the controller 120 first determines if the SpO₂ is in a predetermined range and what the activity level is (rest or exertion/movement), and then generates a signal based on the predetermined range and transmits the signal to the flow regulator 130 to drive the flow regulator 130 to adjust the flow rate of oxygen from the oxygen source corresponding to the predetermined range. The bracelet oximeter or physiologic measurement device determines the activity level of the patient (rest or exertion/movement) and begins the titration of oxygen as below based on the pre-set initial oxygen flow rate determined for rest and activity and prescribed by the provider. These preset starting levels for auto titration by AutO2 (a customized application) for rest and activity are determined based on resting measures and walks in clinic with the provider. Then, these prescribed resting and exertional starting saturations for titration are set in the algorithm. These starting titration levels for rest and exertion can be adjusted as needed as disease progresses and oxygenation worsens.

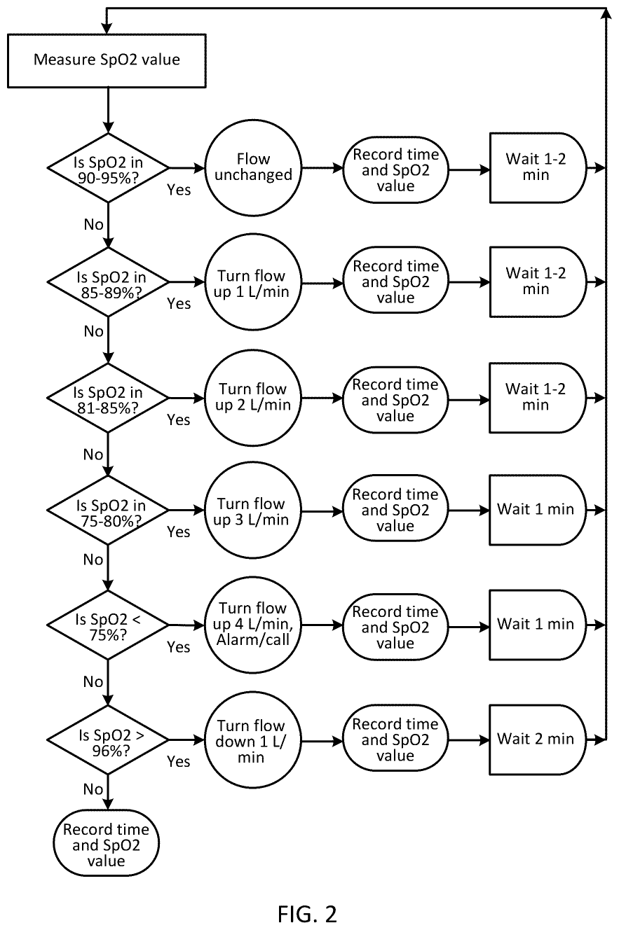

For example, as shown in FIG. 2, when the SpO₂ is in a range of 90-95%, the flow rate remains unchanged, and the SpO₂ is rechecked in a time period of 1-2 minutes;

when the SpO₂ is in a range of 85-89%, the flow rate is adjusted to increase by 1 L/min, and the SpO₂ is rechecked in a time period of 1-2 minutes;

when the SpO₂ is in a range of 81-85%, the flow rate is adjusted to increase by 2 L/min, and the SpO₂ is rechecked in a time period of 1-2 minutes;

when the SpO₂ is in a range of 75-80%, an alarm is triggered, the patient is messaged to sit down/rest, and the flow rate is adjusted to increase by 3 L/min or to a maximum flow rate if 3 L/min is greater than the maximum flow rate, and the SpO₂ is rechecked in a time period of 1-2 min;

when the SpO₂ is less than 75%, an alarm is triggered, the patient is messaged to sit down/rest, and the flow rate is adjusted to increase by 4 L/min or to the maximum flow rate if 4 L/min is greater than the maximum flow rate, and the SpO₂ is rechecked in a time period of 1-2 min; and

when the SpO₂ is greater than 96%, the flow rate is adjusted to reduce by 1 L/min, and the SpO₂ is rechecked in a time period of 2 minutes, which continues until the SpO₂ is maintained in a range of 90-95%.

To ensure accuracy of the pulse oximeter, a regular pulse and repeated SpO₂ is reassessed about 10 seconds later, prior to the above protocol.

In some embodiments, the controller 120 is configured to initiate a call for help, when the patient is in a predetermined physiological status in which the SpO₂ is less than 75% for a predetermined time period, and/or shortness of breath or chest pain is present.

In some embodiments, the controller is configured to average the measured physiological parameters of the patient over a time period of 10-20 sec.

In some embodiments, the controller 120 is configured if more than three alarms occur over a course of a day, the patient is messaged to contact their provider with their oxygen desaturations.

In some embodiments, the controller 120 is configured to store the measured physiological parameters including the SpO₂ and the flow rate over days and weeks in local, or transmit them to a data center, which are accessible by the patient, a professional, and/or a provider.

In one embodiment, the controller 120 is configured to determine whether the patient is in an active state or a rest state using output signals from the at least one accelerometer.

In some embodiments, the controller 120 is a smartphone, a tablet, or any type of smart mobile devices. A customized application with a graphic user interface (GUI) is installed in the smartphone or tablet for operation setting and configuration, user inputs, display of the physiological parameters and notifications, and so on.

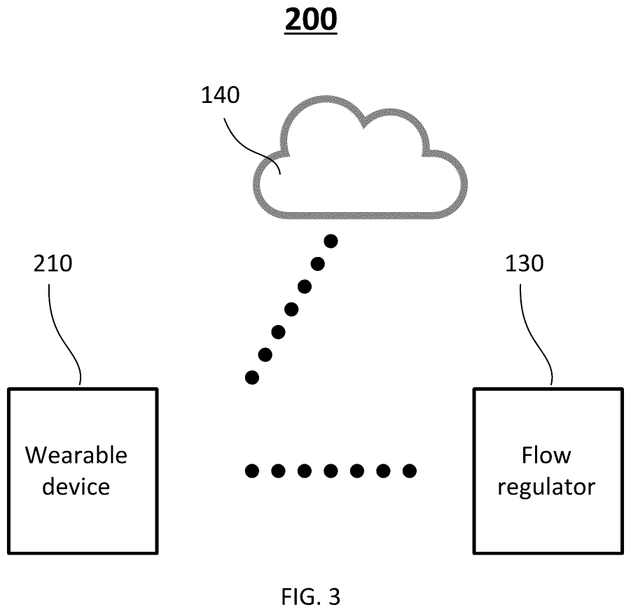

Referring to FIG. 3, a system 200 for automating oxygen monitoring and dosing in real time for a patient on oxygen therapy is shown according to another embodiment of the invention. The system 200 includes a flow regulator 130 coupled to an oxygen source for operably controlling a flow rate of oxygen from the oxygen source to the patient; and a wearable device 210 in communication with the flow regulator 130 and configured to be worn by the patient for measuring physiological parameters of the patient to monitor a physiological status of the patient and controlling operations of the flow regulator 130 to regulate the flow rate of oxygen from the oxygen source for providing an desired amount of oxygen to the patient based on the physiological status of the patient.

In some embodiments, the wearable device 210 comprises at least one pulse oximeter and/or at least one pulse co-oximeter for measuring saturation of oxygen for measuring the SpO₂ of the patient.

In some embodiments, the wearable device 210 further comprises at least one accelerometer for motion measurements so as to determine whether the patient is in an active state or a rest state; and/or at least one temperature sensor for temperature measurements.

In some embodiments, the wearable device 210 further comprises a controller for controlling the operations of the flow regulator 130 based on the physiological status of the patient.

In some embodiments, the wearable device 210 is essentially an integration of a sensor device 110 and a controller 120 of the system 100, and the system 200 has the same functions as that of the system 100, as described above.

According to embodiments of the invention, the system for automating oxygen monitoring and dosing in real time for a patient on oxygen therapy is an automated system and capable of connecting to industry standard oxygen concentrators/tanks, sensing patient's oxygen saturation in real time, and adjusting the flow rate according to the patient's needs to maintain appropriate oxygenation, reducing oxygen waste and costs, and eliminating the hassle of oxygen titration. The system creates a closed loop system that makes adjustments of oxygen based changes in the skin sensors, which yields profoundly better results in patient outcome.

In one aspect, the invention also relates to a method for automating oxygen monitoring and dosing in real time for a patient on oxygen therapy. The method comprises continuously measuring the patient's physiological parameters such as SpO₂ by a wearable device worn by the patient and adjusting a flow rate of oxygen from an oxygen source, by a flow regulator coupled to the oxygen source, for providing an desired amount of oxygen to the patient based on the SpO₂ and activity level.

In some embodiments, the method further comprises generating a signal corresponding to the SpO₂ and activity level, by the wearable device or a mobile device in communication with the wearable device and the flow regulator; and transmitting the signal to the flow regulator to cause the flow regulator to adjust the flow rate of oxygen from the oxygen source corresponding to the SpO₂, by the wearable device or the mobile device. For example, when the SpO₂ is in a range of 90-95%, the flow rate remains unchanged, and the SpO₂ is rechecked in a time period of 1-2 minutes; when the SpO₂ is in a range of 85-89%, the flow rate is adjusted to increase by 1 L/min, and the SpO₂ is rechecked in a time period of 1-2 minutes; when the SpO₂ is in a range of 81-85%, the flow rate is adjusted to increase by 2 L/min, and the SpO₂ is rechecked in a time period of 1-2 minutes; when the SpO₂ is in a range of 75-80%, an alarm is triggered, the patient is messaged to sit down/rest, and the flow rate is adjusted to increase by 3 L/min or to a maximum flow rate if 3 L/min is lower than the maximum flow rate, and the SpO₂ is rechecked in a time period of 1 min; when the SpO₂ is less than 75%, an alarm is triggered, the patient is messaged to sit down/rest, and the flow rate is adjusted to increase by 4 L/min or to the maximum flow rate if 4 L/min is lower than the maximum flow rate, and the SpO₂ is rechecked in a time period of 1 min; and when the SpO₂ is greater than 96%, the flow rate is adjusted to reduce by 1 L/min, and the SpO₂ is rechecked in a time period of 2 minutes, which continues until the SpO₂ is maintained in a range of 90-95%. The starting point of flow rate for titration is based on activity level as determined by the wearable device.

In some embodiments, the method further comprises initiating a call for help (e.g., 911 call), when the patient is in a predetermined physiological status in which the SpO₂ is less than 75% for a predetermined time period, and/or shortness of breath or chest pain is present.

In some embodiments, the method further comprises storing the measured physiological parameters including the SpO₂ and the flow rate over days and weeks in local, and/or transmitting them to a data center.

In some embodiments, the method further comprises noticing the patient to contact their provider with their oxygen desaturations if more than three alarms occur over a course of a day. In some embodiments, the method further comprises generating a report that include the physiological parameters, a flow rate, alarms, time, and locations.

In one aspect of the invention, the system for automating oxygen monitoring and dosing in real time for a patient on oxygen therapy comprises a wearable sensor device configured to measure oxygen saturation levels of the patient; track physical activity of the patient; and transmit real-time oxygen saturation and activity data; and a smart flow regulator operably coupled to an oxygen source and configured to receive the transmitted data from the wearable sensor device; implement an adaptive control algorithm to determine an appropriate oxygen flow rate based on the received oxygen saturation and activity data; and automatically adjust a flow rate of oxygen delivered from the oxygen source to the patient in real time.

In some embodiments, the wearable sensor device comprises at least one pulse oximeter and at least one accelerometer integrated within a wearable housing configured to be worn on a wrist, arm, ankle, or other body part of the patient.

In some embodiments, the system further comprises a controller configured to coordinate data exchange, execute the adaptive control algorithm, and maintain the oxygen saturation level within a predetermined target range.

In some embodiments, the smart flow regulator comprises a stepper motor mechanically coupled to a flow adjustment member of the oxygen source; and a driving circuit configured to receive control signals from the controller and drive the stepper motor to adjust the oxygen flow rate accordingly.

In some embodiments, the smart flow regulator is configured for digital or mechanical coupling with a home or portable oxygen concentrator or tank.

In some embodiments, the smart flow regulator includes an emergency override mechanism permitting manual flow rate adjustment in the event of communication failure.

In some embodiments, the adaptive control algorithm comprises a proportional-integral-derivative (PID) control logic configured to smooth variations in oxygen saturation and prevent overcorrection.

In some embodiments, the controller operably averages measured oxygen saturation data over a predetermined interval of 10-20 seconds prior to calculating flow rate adjustments.

In some embodiments, the controller is configured to classify the physical activity level of the patient into resting and active states based on actigraphy data, and to apply corresponding baseline oxygen flow rates for each state.

In some embodiments, the baseline oxygen flow rates for resting and active states are pre-configured by a healthcare provider.

In some embodiments, the controller operably executes a titration protocol comprises maintaining the current flow rate when oxygen saturation is between 90% and 95%; increasing the flow rate when oxygen saturation falls below 90%; and reducing the flow rate when oxygen saturation exceeds 95%.

In some embodiments, the controller is further configured to trigger an alarm or visual notification when oxygen saturation falls below a predetermined threshold; and prompt the patient to rest or automatically contact emergency services if the low oxygen condition persists for a set time period.

In some embodiments, the controller is configured to generate daily or weekly reports of oxygen saturation, activity level, and oxygen flow data accessible to a healthcare provider via a secure portal.

In some embodiments, communication between the wearable sensor device, the controller, and the smart flow regulator is achieved wirelessly using at least one of Bluetooth®, Wi-Fi, or cellular communication protocols.

In some embodiments, the controller is integrated in the smart flow regulator, the wearable sensor device, or an external device.

In some embodiments, the external device comprises a smartphone or tablet executing a software application configured to receive oxygen saturation and activity data from the wearable sensor device; transmit flow control signals to the smart flow regulator; display real-time physiological and system information; and store data locally or on a remote server for review by healthcare providers.

In some embodiments, the wearable sensor device is further configured to monitor additional physiological parameters including heart rate, respiratory rate, skin temperature, and blood pressure, and wherein the controller adjusts oxygen delivery based on one or more of said parameters.

In another aspect of the invention, the method for automating oxygen monitoring and dosing in real time for a patient on oxygen therapy comprises measuring oxygen saturation levels of the patient with a wearable sensor device; tracking physical activity of the patient with the wearable sensor device; transmitting real-time oxygen saturation and activity data from the wearable sensor device to a controller; processing the transmitted data using an adaptive control algorithm executed by the controller to determine an oxygen flow rate appropriate for the patient's physiological and activity state; and automatically adjusting a smart flow regulator operably coupled to an oxygen source to deliver oxygen at the determined flow rate.

In some embodiments, the method further comprises classifying the activity level of the patient as resting or active based on actigraphy data, and applying corresponding baseline oxygen flow rates for each state.

In some embodiments, the adaptive control algorithm comprises a proportional-integral-derivative (PID) feedback system configured to incrementally adjust oxygen flow based on averaged oxygen saturation readings over a time window of 10-20 seconds.

In some embodiments, the method further comprises maintaining the oxygen saturation level within a target range of 90% to 95% by increasing the flow rate when oxygen saturation falls below the target range; and decreasing the flow rate when oxygen saturation exceeds the target range.

In some embodiments, the method further comprises triggering an alarm and notifying the patient to rest or seek assistance when the oxygen saturation level falls below a critical threshold for a predetermined time period.

In some embodiments, the method further comprises storing oxygen saturation, activity, and flow rate data locally or transmitting the data to a cloud database accessible to a healthcare provider.

In some embodiments, the smart flow regulator comprises a stepper motor operably connected to a mechanical or digital flow control member of an oxygen concentrator or tank.

In some embodiments, the method further comprises averaging oxygen saturation readings over a predetermined interval to account for physiologic delay in oxygen uptake prior to adjusting the flow rate.

In some embodiments, the method further comprises automatically initiating an emergency call or provider alert if oxygen saturation remains below a critical level for a predetermined duration.

In some embodiments, communication between the wearable sensor device, the controller, and the smart flow regulator is performed via a wireless communication protocol selected from the group consisting of Bluetooth®, Wi-Fi, and cellular communication.

In some embodiments, the method further comprises generating and displaying in a software application a graphical summary of oxygen saturation trends, activity levels, and corresponding oxygen flow rates for review by a healthcare provider.

In some embodiments, the adaptive control algorithm further utilizes historical patient data to predict oxygen needs and pre-emptively adjust the flow rate during anticipated periods of exertion.

In yet another aspect of the invention, the method for personalized oxygen delivery to a user on oxygen therapy comprises continuously monitoring an oxygen saturation level of the user with a wearable oxygen sensor; tracking, in real time, a physical activity level of the user using an activity sensor integrated with or communicatively coupled to the wearable oxygen sensor; dynamically adjusting a flow rate of oxygen delivered from an oxygen source to the user in response to at least one of the detected physical activity level; and the measured oxygen saturation level; incrementally increasing or decreasing the oxygen flow rate at predetermined intervals in accordance with an adaptive titration protocol, thereby maintaining the oxygen saturation level within a predetermined range; and reassessing the oxygen saturation data after each incremental flow rate adjustment to determine whether further adjustment is required.

In some embodiments, the reassessment of oxygen saturation occurs approximately every two minutes following each flow rate adjustment.

In some embodiments, the step of incrementally adjusting the oxygen flow rate comprises increasing the flow rate by predetermined increments when the oxygen saturation falls below a threshold level and decreasing the flow rate when the oxygen saturation exceeds an upper limit.

In some embodiments, the method further comprises classifying the user's activity level as one of a resting state or an active state, and initiating adjustment of the flow rate from respective baseline oxygen flow rates associated with the classified state.

In some embodiments, the dynamically adjusting step is performed automatically by a controller executing an adaptive algorithm configured to interpret both oxygen saturation and activity data.

In some embodiments, the adaptive algorithm comprises a proportional-integral-derivative (PID) feedback control that smooths variations in oxygen saturation and compensates for physiological delay in oxygen uptake.

In some embodiments, the method further comprises averaging measured oxygen saturation values over a predetermined time window of about 10-20 seconds prior to each reassessment.

In some embodiments, the method further comprises triggering an alert when the oxygen saturation falls below a predetermined critical threshold for longer than a preset duration.

In some embodiments, the incremental adjustment of the oxygen flow rate occurs in discrete steps of approximately 1 liter per minute (L/min) or less.

In some embodiments, the predetermined reassessment interval and adjustment increments are configurable by a healthcare provider via a software interface.

In some embodiments, the method further comprises storing oxygen saturation, activity level, and flow rate data in a local or cloud-based memory for review by a healthcare provider.

In some embodiments, the dynamically adjusting step is based solely on detected physical activity when valid oxygen saturation data are unavailable or outside a confidence threshold.

In some embodiments, the reassessment interval and incremental flow changes are coordinated such that no flow adjustment is performed until a stable oxygen saturation value has been confirmed over a preceding assessment window.

In some embodiments, the method further comprises predicting upcoming oxygen demands based on historical activity data of the user and preemptively adjusting the oxygen flow rate to maintain desired oxygen saturation levels.

It should be noted that all or a part of the steps according to the embodiments of the invention is implemented by hardware or a program instructing relevant hardware. Yet another aspect of the invention provides a non-transitory tangible computer-readable medium storing instructions which, when executed by one or more processors, perform the above method for automating oxygen monitoring and dosing in real time for a patient on oxygen therapy. The computer executable instructions or program codes enable a computer or a similar computing system to complete various operations in the above disclosed method for privilege management. The storage medium/memory may include, but is not limited to, high-speed random access medium/memory such as DRAM, SRAM, DDR RAM or other random access solid state memory devices, and non-volatile memory such as one or more magnetic disk storage devices, optical disk storage devices, flash memory devices, or other non-volatile solid state storage devices.

Without intent to limit the scope of the invention, examples and their related results according to the embodiments of the invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Flow Regulator (Smart Knob)

The flow regulator in one embodiment of the invention is a mechanical or digital smart flow knob that is operably engaged with a standard oxygen concentrator/tank for automatically adjusting the oxygen flow rate from the oxygen tank to the patient according to the patient's physiological status that is determined by the measured physiological parameters. Since the flow rate on most oxygen concentrators is controlled by the twisting of a knob, a general design that can change its size can be created to attach to the knob and then the device can be manually calibrated according to the sensitivity of the oxygen concentrator.

The flow regulator has a stepper motor; a connection member engaged with the stepper motor and the oxygen source such that rotation of the stepper motor drives the connection member to rotate, which in turn adjusts the flow rate of oxygen from the oxygen source; and a driving circuit in communication with the controller and the stepper motor for receiving the signal from the controller and driving the stepper motor with the signal so as to adjust the flow rate of oxygen accordingly.

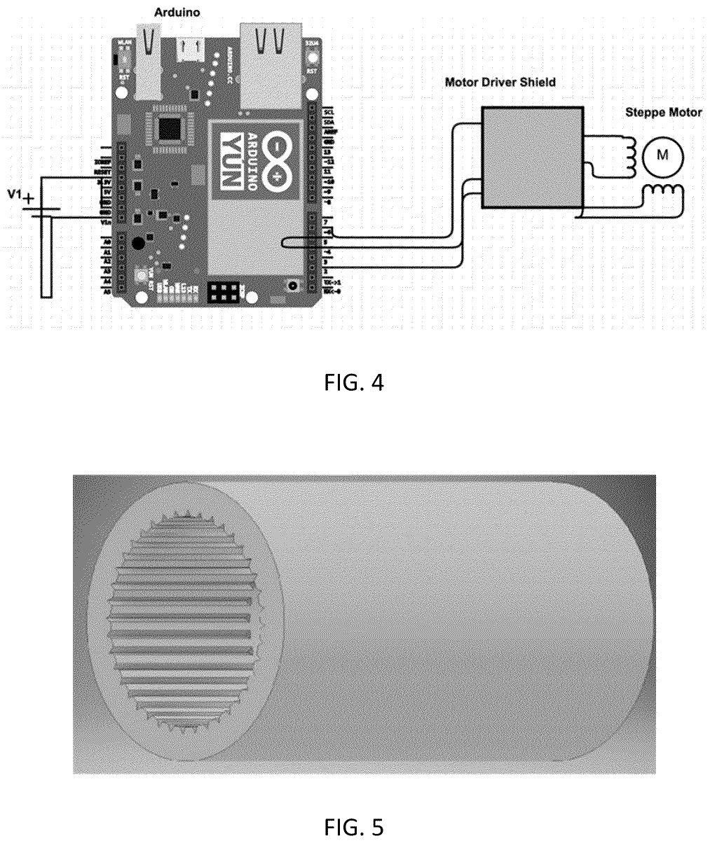

As shown in FIG. 4, a schematic circuit diagram used for the mechanistic driver within the flow regulator is shown according to one embodiment of the invention. The circuit includes an Arduino board, a breadboard, assorted wires, a Bluetooth driver, a stepper motor driver, and a stepper motor. The stepper motor is engaged with a connection member that is in turn connected to an oxygen tank/concentrator. The connection member is configured to allow the flow regulator to transfer motor's rotation to a knob of the oxygen tank.

As shown in FIG. 5, in one embodiment the connection member is a nylon cylinder with ridges on the interior. The ridges on the cylinder match those on the oxygen tank allowing it to fit directly on to the oxygen tank knob. The other side of the cylinder connects to the stepper motor thereby transferring the inertia from the stepper motor to the oxygen tank knob.

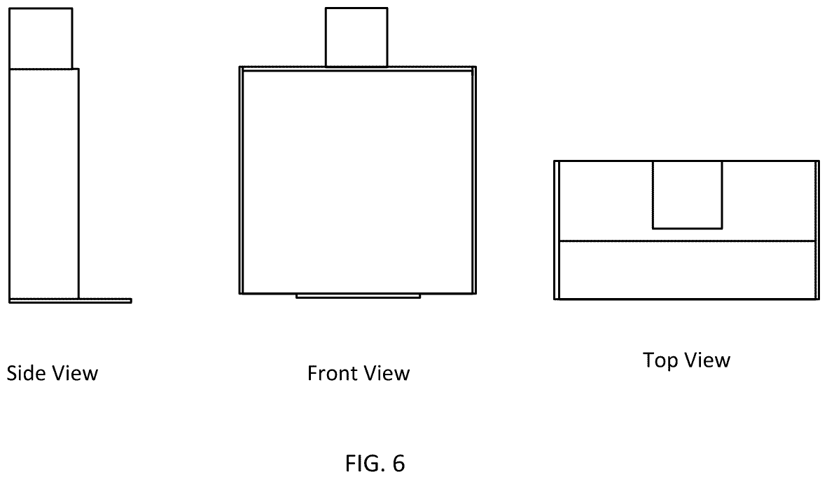

The mechanistic driver is contained in an acrylic box atop an acrylic shield that can be placed next to the oxygen tank. The acrylic structure is shown FIG. 6. The top section of the side and front views shows the box where the mechanistic driver and all of its components are placed. The rest of the structure is placed near the oxygen tank and provide support for the mechanistic driver.

The flow regulator (device) is able to attach to and alter flow on existing oxygen concentrators, meaning that no new concentrators need to be made for this device. The user also has the ability to remove the device if errors occur and the system is able to be calibrated to the sensitivity of the oxygen concentrator it is being used on.

In other embodiments, some portable oxygen concentrators and home oxygen concentrators are digital and electronically driven instead of driven or adjusted by a rotary knob. For digital concentrators, accessibility for adjustment is included in this concept. For example, in some embodiments, the flow regulator is configured such that the flow rate of the digital concentrators is electronically driven through digital means. In other embodiments, the flow regulator comprises digital means for adjusting the flow rate of the digital concentrators electronically.

### Example 2

### Algorithm and Application Protocol

According to embodiments of the invention, the algorithm and application protocol provides improved oximeters by using wrist pulse oximetry, and multi wavelength pulse co-oximetry, instead of finger and earlobe pulse oximetry, and averaging the reading from the pulse oximeter over about 10-20 sec.

The algorithm and application protocol also provides the oxygen flow rate adjusted according to the patient's SpO₂ per protocol with feedback by, for example, a proportional-integral-derivative (PID) system with mass flow regulator allowing for up to 20 L/min. Reading from the pulse oximeter is averaged over about 10-20 sec.

The algorithm and application protocol allows time for accurate reading/discriminating error and artifact. Each circulation of a red blood cell (RBC) takes about 60 seconds. SpO₂ readings sent to adjust the flow rate too close together do not allow time for circulation or accurate assessment with adjustment of the flow rate. Most devices capture the SpO₂ signal every second with rapid adjustment of the flow rate. This can result in over and under titration of oxygen in patients. Most automated systems in the public space show up to 22% desaturation during the measured time of use.

The algorithm and application protocol also allows to set the parameter of starting flow rates for O2 at rest and with exertion, so as to minimize the time needed to get to the final best flow rate with fewer O2 flow rate titrations to keep saturations at 90% or better with rest or exertion. Rest to exertion and exertion to rest trigger changes in the actigraphy that is transmitted to the algorithm.

The algorithm and application protocol has the ability of the pulse oximetry to send a signal directly to a phone and the flow regulator. If the phone battery dies, the signal can divert to the flow regulator so that oxygen titration is not interrupted.

The algorithm and application protocol can also alert patients if signals are lost from wearables or phone, low sustained saturations occur for 60 seconds or longer, and/or high sustained pulse rate (>140) and low pulse rate (<40).

The algorithm and application protocol has the ability for providers to set alert parameters and resting/exertional flow rates, message the patient/the primary care physician (PCP)/pulmonologist/pulmonologist's triage nurse if the patient's flow rates and oxygen needs are increasing over the past week or set time period. These parameters with messaging endpoint can be set by providers. Data acquisition of needed flow rates and saturation occur over the prior week or month via online database-similar to CPAP downloads of compliance and AHI data.

The algorithm and application protocol also provides security with password protected entry of prescribed parameters, and password protection for data acquisition by providers.

Most devices do not take error or artifact of the oximeter into account. Multi wavelength pulse co-oximetry is utilized to improve sensing. The reading from pulse oximeter should be averaged over 10-20 sec before use in algorithm to avoid reading errors with artifact and drop out of pulse and SpO₂. The wearable sends SpO₂ and actigraphy data to the application on the smartphone. The oxygen flow rate is adjusted according to the SpO₂ per protocol within the app with feedback. Most devices capture the SpO₂ every second which is too quickly for human physiology to respond. Most automated systems show % desaturation of up to 22% of the testing time. This device takes the risk of artifact and human physiologic delay into account in the algorithm.

A PID system with mass flow regulator allows for adjustment of flow rate up to 20 L/min.

For the flow regulator (Smartknob), parameters of the starting oxygen flow rate are set at rest and with exertion. For example: 2 L/min at rest and 6 L/min with exertion. Set resting and exertional flow rates as a starting point for oxygen titration allow for large jumps to occur with changes in activity level up or down with smaller titrations from the set resting and exertional flow rates. This will minimize the time need to get to the final best flow rate w fewer titration keep saturations in 90s with exertion or rest. Rest to exertion and exertion to rest triggers recording of movement or rest with actigraphy incorporated in the wearable. App sends signal to Smartknob on oxygen regulator to adjust flow rate.

In some embodiments, the provider sets the “resting” and “exertional” or “activity” flow rate. The App algorithm begins titration from set flow rates. Actigraphy identifies if patient is at “rest” or “exertion”. If at rest or sitting, titration begins at set “resting” flow rate. If moving, active or exerting, titration begins at set “activity” flow rate. One exemplary example of the algorithm is as follows:
If the SpO₂ is in the range of 90-95%, then the flow rate remains unchanged.
If the SpO₂ is the range of 87-89%, then the flow rate is increased by 1 L/min.
If the SpO₂ is the range of 83-86%, then the flow rate is increased by 2 L/min.
If the SpO₂ is the range of 79-82%, then the flow rate increases by 3 L/min and trigger alarm noting low oxygen saturation.
If the SpO₂ is <79%, the flow rate increases to max flow with audible alarm advising patient to rest, call for assistance or 911.
If the SpO₂>95%, then the flow rate decreases by 1 L/min.

The SpO₂ is measured with a moving window of 10-20 seconds that identifies a consistent pulse (to ensure good signal) and averages continuously over the prior 10-20 seconds. The SpO₂ is continuously detected with the prior 10-20 seconds averaged by the AutO2 app.

### Example 3

Auto2 App with User Interface

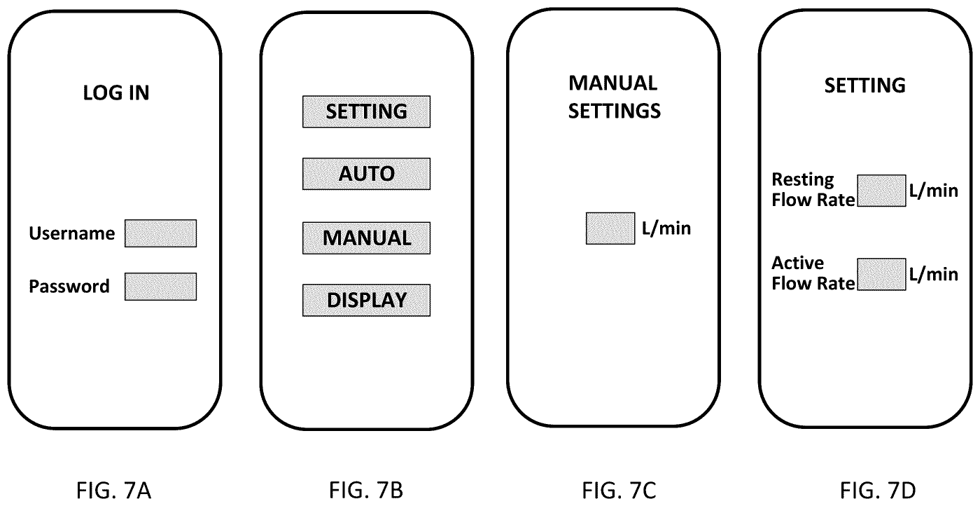

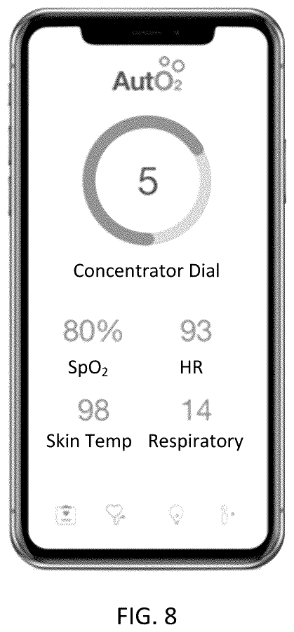

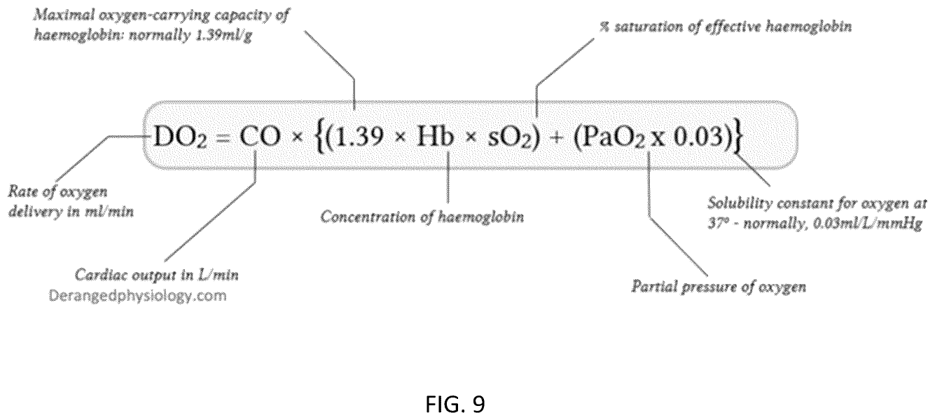

FIGS. 7A-7D and 8 show one embodiment of a customized application (AutO2 app) with a GUI installed in the smartphone for operation setting and configuration, user inputs, display of the physiological parameters and notifications, and so on.

FIGS. 7A-7D show a few of pages of the AutO2 app for operation setting and configuration and user inputs, as an example. It should be appreciated that the AutO2 app can also be presented in other forms. The first page of the AutO2 app show in FIG. 7A is for logging in, where username and password are required to initiate the AutO2 app. The new few pages shown in FIGS. 7B-7D are for user inputs of the blood O₂ saturation level in addition to manually inputting the oxygen liter flow rate the patient requires.

For example, the second page shown in FIG. 7B lists a number of confirmations, such as “SETTING”, “AUTO”, “MANUAL” and “DISPLAY”. When “AUTO” is selected, the application will automatically set resting and exertional flow rates based on the actigraphy reading of rest vs exertion. When “MANUAL” is selected, the application will allow the patient to set the flow rate at this moment, as shown in FIG. 7C. This is a safety hatch so that if the patient desaturates and needs to adjust to a maximal flow rate of oxygen, they can. When “SETTING” is selected at the fourth page shown in FIG. 7D, the provider can set “Resting Flow Rate”—flow rate needed to keep saturations in the 90's at rest, and “Active Flow Rate”—flow rate needed to keep saturations in the 90's during activity, like walking. In some embodiment, scrolling wheel with numbers up to 20 L/min is adapted for setting resting and active flow rates. The app should sense a transition to activity by the actigraphy monitor on the wearable device and increase the flow rate to the active level to allow a quicker adjustment of the flow rate to keep saturations in the 90's with activity. When “DISPLAY” is selected, the application will display SpO₂, HR, Skin Temperature, Respiratory rate and Flow rate. One exemplary display is shown in FIG. 8.

In conclusion, the system for pulse oximeter saturation monitors and API/Bluetooth devices to adjust a knob (like the knob on an oxygen regulator) are disclosed. The unique feature of the system and method is the combination of monitoring coupled with a unique protocol adapted into an app to send the correct flow rate signal via Bluetooth to the oxygen regulator once the oxygen saturation has been interpreted by the pulse oximeter. No one has linked these elements with a unique smart oxygen auto adjusting protocol. This unique know how is the link between measurement of oxygen saturation and deployment of the correct dosing of oxygen. The system provides automated oxygen monitoring and dosing in real time allowing patients wearing oxygen to no longer need to constantly think about adjusting their oxygen flow rates as their activity level changes.

Other applications of the device include deployment in the hospital environment. This is perhaps an even more important application for patient safety. Presently, patients on oxygen in hospitals are adjusted by the nurse who may notice alarms for low oxygen levels or tachycardia and respond by leaving one patient's room and moving to the one with alarms sounding. Due to location and the duties the nurse is performing with another patient, a time delay in adjustment of oxygen occurs. A real time device that sounds the alarm while adjusting the oxygen per software protocol to the flow rate needed by the patient to maintain normoxia would correct hypoxia sooner while avoiding lost time and the numerous complications of hypoxia most notably arrythmia with cardiopulmonary arrest in the acutely ill patient.

As we transition to telehealth visits in this post CoVID-19 era, data on oxygenation and respiratory status in the home is lacking. Having objective data from the home via this device could provide an additional element of objective data to these visits and possibly provide more helpful information than our six minute walk tests or other clinic walk tests can provide.

### Example 4

### Actigraphy on the Wearable Identifies Movement or Rest for Patient

With rest, patients need lower flow rates and with exertion need higher flow rates to achieve oxygen saturations in the 90s. When the wearable identifies movement and notifies the app of movement, a signal for the flow rate to jump to the activity/exertional/movement flow rate as prescribed by the MD occurs with further titration off this higher flow rate. The reverse occurs with a change from movement to rest with a drop to the prescribed resting flow rate. Hence, the provider prescribes the resting and exertional flow rate of oxygen via a walk test in clinic and the device is set to these parameters. This allows for titration from the set resting flow rate and from the set exertional flow rate. This titration strategy provides a quicker adjustment to the needed higher flow rate with activity and a shorter time for a climb in flow rate and oxygen saturation to occur.

If the patient is at rest, the oxygen flow rate starts at the set flow rate for rest. The SpO₂ is recorded for 5 minutes after starting up or changing from activity/movement. Titration then occurs based on the average SpO₂ over the last 30 seconds according to the chart.

If the patient begins to move by actigraphy, the flow rate jumps immediately to the exertional flow rate and records saturation×5 minutes. Titration of the oxygen flow rate then occurs according to SpO₂ value from wearable by chart with assessments at 2 minute intervals following an adjustment of flow rate.

If the patient returns to rest, the SpO₂ is measured for 5 minutes and then the flow rate returns to the resting flow rate. The oxygen flow rate is titrated according to the chart and reassessed after 2 minutes if the flow rate is changed.

Titration is based on (1) the time it takes the heart to pump oxygenated blood throughout the body which is about 60 seconds. An allowance of 2 minutes gives generous time for slower circulation as could occur in heart failure, and/or (2) a longer time of 5 minutes for measurement and oxygen saturation equilibration is given when patients change from rest to movement or movement to rest. This allows for cardiac output to increase or decrease accordingly and stabilize prior to oxygen adjustment. Cardiac output is a determinant of oxygen delivery.

### Example 5

### Real-Time Monitoring and Automated Adjustment Device for Oxygen Therapy: Improving Accuracy and Quality of Life for Patients

Long-term oxygen therapy is a standard of care for chronic hypoxic respiratory failure. Over 1.5 million Americans are prescribed oxygen, yet patients are left to self-dose their oxygen with limited guidance. Inexact dosing can result in adverse effects, including shortness of breath, reduced endurance, and more serious conditions such as pulmonary hypertension or ischemia.

In this exemplary work, we disclose a novel automated oxygen delivery device, AutO2, that promises to transform the lives of hypoxic patients. With the unique algorithm, the oxygen delivery device improves oxygen titration and provides a seamless and personalized oxygen therapy experience. This groundbreaking device has the potential to enhance patients' quality of life, improves health outcomes, and transforms oxygen therapy. The device works by (1) connecting a wearable oximeter via Bluetooth connection to the phone application, (2) processing the oximeter data with the proprietary algorithm to determine the appropriate oxygen flow rate, and (3) sending Bluetooth® controls the smart flow regulator to automatically adjust to those levels.

The outcomes from this work include data that supports the central hypothesis that automated oxygen titration significantly improves patients' oxygenation and quality of life and a functional application and algorithm that is ready for full clinical trials with the most capable oximeter. The data collected during this phase serve as the basis for the design and prototype of the smart flow regulator. For a fully developed device, it has tangible benefits in the following areas:

Health: With improved oxygenation, individuals experience improvements in endurance, shortness of breath, and quality of life.

Economic: Disease progression can be identified and treated earlier than ever before, leading to reduced healthcare system utilization. Our device enables more efficient use of oxygen which reduces waste. Oxygen use data can also be collected and prove integral to future Medicare policy.

Societal: Respiratory illnesses are rising at a significant pace. Our device addresses the needs of millions of people, allowing them to engage more fully in their personal lives and community.

### Oximeter Parameter Testing

Rationale: Because of shortfalls of wrist oximeters that were identified in preliminary testing, Respiro must consider other products. We test new wearable oximeters to discern the device that collects the most accurate data in a real-world setting. To accomplish this, we use specific Device Selection Criteria to screen oximeter candidates on the market, noting improvement over the common issues with wrist oximeters and establishing minimum acceptable parameters for critical measurements such as continuous SpO₂ accuracy. We work with companies to explore partnership opportunities and determine what engineering changes can be made to their product. Once the top two devices are determined, the devices are purchased for testing in a prospective human feasibility study. The devices are placed in a real-world head-to-head comparison to understand the oximeters' performance and gain user feedback. In the study, participants from the Vanderbilt ILD program wear one oximeter for one week and the other oximeter for one week, allowing participants to act as their own control. Participants are asked to complete this observational period followed by useability surveys. Insights from the data collected during the trial from the oximeters associated application and useability surveys are used to select the best performing oximeter for further testing.

Methodology: Two examples of wrist oximeter alternatives are the Oxiwear Ear and Wellue O₂ Ring oximeters. By placing the oximeter on the ear or finger, both companies claim to overcome motion artifacts and skin tone/thickness limitations associated with wrist oximeters. Both oximeters are also an example of a product that needs upgrades/alterations to meet our final products' engineering requirements (i.e., actigraphy and extended battery life).

Potential Pitfalls and Alternative Strategies: It is possible that recorded patient preferences and oximeter performance data does not allow a clear winner to be identified. In anticipation of such occurrence, we maintain a full list of acceptable oximeters to conduct future tests, if necessary. If no device meets the requirements, we are still able to move forward with the rest of our project but coordinate with manufacturers or look to produce our own device. We chose to recruit patients from the Vanderbilt ILD program because it is a nationally and internationally respected clinical trials center that encompasses a 7-state referral base and sees over 20 new ILD patients per week.

### Phone Application Enhancements

Rationale: We improve the overall functionality of our phone application and make it compatible with the oximeter we selected for testing, integrating our proprietary algorithm.

Methodology: this application is compatible with the new oximeter, integrates the algorithm, and verifies/tests the upgrades.

Update User Interface: Redesign the user interface to cater to our target market which consists of individuals who are less tech-savvy and may have visual or tactile impairments. Create a password protected provider workspace and improve graphical representation of data within the app to provide insights on oxygen saturation events and trends.

Oximeter Compatibility: Ensure the selected oximeter can function properly with the application, maintain connectivity, and provide required measurement data.

Algorithm Integration: create algorithm code within the application to notify patients of low oxygen saturation and the ability to send output controls to our eventual prototype.

Verification and Testing: Test and verify the ability of the algorithm to adjust O₂ flow rate to SpO₂ fluctuations.

Once these steps are complete, we conduct a prospective human study with ILD patients in the clinical trials program at VUMC. 20 ILD patients and at least 5 pulmonary providers (MD, DO, midlevel providers) are asked to wear the device with connected application on a research cell phone for a total of 96 hours. For the first 48 hours notifications are turned off and for the next 48 hours they are turned on. Participants are instructed not to make changes to their oxygen flow rate without contacting their provider. The purpose of this testing is to gather data, verify functionality, and determine what changes, if any, need to be made in the application source code. Patient and physician feedback are also compiled to uncover preferences on the user interface and data presentation of the application. This information is critical to determine Respiro's focus for future iterations of the software and algorithm.

Potential Pitfalls and Alternative Strategies: Unforeseen complexity of integrating the algorithm to the application build. To decrease the likelihood of this occurring, Respiro has allocated a portion of its current resources to establish the groundwork with our technical consultant before receiving SBIR funding.

### Deliverables

Data and written reports of results relating to the performance of each oximeter tested and patient feedback. This information informs our decision to move forward with a commercial product and/or the engineering updates that need to be made to work with our product.
1) Fully operational phone application with integrated algorithm.
2) Data and written reports of performance of the phone application with the selected oximeter and human research study. Patient and physician feedback are compiled to uncover preferences on the user interface and data presentation that need to be made prior to the next stage of development.

The top oximeter candidate which provides superior measurement accuracy, least measurement dropout rate, and comfort is selected, and additional devices are purchased for use.

Upgraded software with an improved user interface, tested and functioning algorithm capable of sending SpO₂ adjustment signals to a smart flow device prototype.

### Competitive Landscape & Advantage

The AutO2 proprietary algorithm is what sets it apart from competitors. Our patent-pending algorithm processes the data of real time SpO₂, vitals, and actigraphy from the patient's wearable oximeter. The algorithm considers the SpO₂ (averaged over 20 seconds) and adjusts the flow rate as needed from this baseline level. When a patient exerts themselves, the oximeter senses the physical activity which signals the algorithm to adjust the smart flow regulator to the flow rate prescribed for activity. Titrating oxygen from these prescribed levels for rest and activity allows the device to reach a flow rate that maintains the oxygen saturation of the patient in the 90-99% range in a shorter amount of time than competitors and provides more accurate oxygen dosing. Without this parameter in our algorithm, patients might never reach the needed physiologic SpO₂ in the 90s before changing activity levels again. Unlike other devices, AutO2 aims to treat all forms of hypoxic respiratory failure. The real-time data collection that our device will provide is paradigm changing. For the first time, providers will be able to view a patient's oxygenation over set timeframes to assess whether their prescription is working adequately. Graphical representations of SpO₂ and actigraphy can be viewed in just seconds, adding efficiency to clinic visits and improving the dosing of oxygen. Remote patient monitoring will be a focus point for future stages of development.

Our AutO2 application demonstrates immense viability in the market with over 1.5 million patients on oxygen therapy in the United States. Furthermore, the oxygen therapy market is projected to grow at a CAGR of 6.2% from 2023-2030. The product's seamless integration into patients' daily lives makes it a compelling choice for those seeking a more convenient and effective oxygen therapy solution. By automating oxygen delivery, the device represents a significant step forward in improving the comfort, independence, and overall well-being of our customers: patients and caretakers alike. The applicability of AutO2 extends to hospitals, research facilities, outpatient care, and the Department of Defense.

The novel device improves health outcomes based on the striking similarity between two technologies that were approved by the FDA in the 1990's—the continuous glucose monitor and the auto-titrating continuous positive airway pressure machine (aCPAP). These devices revolutionized the treatment of diabetes and sleep apnea, respectively, and work similarly to our device. Studies on the glucose monitor uncovered that systems that provide immediate feedback to patients and decision support tools demonstrated superior outcomes compared to self-monitored blood glucose alone. The auto-titrating CPAP machine which uses computer algorithms and sensors, eliminates apneic and hypopneic events, and improves sleep quality and cognitive function.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the invention pertains without departing from its spirit and scope. Accordingly, the scope of the invention is defined by the appended claims rather than the foregoing description and the exemplary embodiments described therein.

What is claimed is:

1. A system for automating oxygen monitoring and dosing in real time for a patient on oxygen therapy, comprising:
a wearable sensor device configured to measure oxygen saturation levels of the patient; track physical activity of the patient; and transmit real-time oxygen saturation and activity data; and
a smart flow regulator operably coupled to an oxygen source and configured to receive the transmitted data from the wearable sensor device; implement an adaptive control algorithm to determine an appropriate oxygen flow rate based on the received oxygen saturation and activity data; and automatically adjust a flow rate of oxygen delivered from the oxygen source to the patient in real time.

2. The system of claim 1, wherein the wearable sensor device comprises at least one pulse oximeter and at least one accelerometer integrated within a wearable housing configured to be worn on a wrist, arm, ankle, or other body part of the patient.

3. The system of claim 1, further comprising a controller configured to coordinate data exchange, execute the adaptive control algorithm, and maintain the oxygen saturation level within a predetermined target range.

4. The system of claim 3, wherein the smart flow regulator comprises:
a stepper motor mechanically coupled to a flow adjustment member of the oxygen source; and
a driving circuit configured to receive control signals from the controller and drive the stepper motor to adjust the oxygen flow rate accordingly.

5. The system of claim 3, wherein the smart flow regulator is configured for digital or mechanical coupling with a home or portable oxygen concentrator or tank.

6. The system of claim 3, wherein the smart flow regulator includes an emergency override mechanism permitting manual flow rate adjustment in the event of communication failure.

7. The system of claim 3, wherein the adaptive control algorithm comprises a proportional-integral-derivative (PID) control logic configured to smooth variations in oxygen saturation and prevent overcorrection.

8. The system of claim 3, wherein the controller operably averages measured oxygen saturation data over a predetermined interval of 10-20 seconds prior to calculating flow rate adjustments.

9. The system of claim 3, wherein the controller is configured to classify the physical activity level of the patient into resting and active states based on actigraphy data, and to apply corresponding baseline oxygen flow rates for each state.

10. The system of claim 8, wherein the baseline oxygen flow rates for resting and active states are pre-configured by a healthcare provider.

11. The system of claim 3, wherein the controller operably executes a titration protocol comprises:
maintaining the current flow rate when oxygen saturation is between 90% and 95%;
increasing the flow rate when oxygen saturation falls below 90%; and
reducing the flow rate when oxygen saturation exceeds 95%.

12. The system of claim 3, wherein the controller is further configured to:
trigger an alarm or visual notification when oxygen saturation falls below a predetermined threshold; and
prompt the patient to rest or automatically contact emergency services if the low oxygen condition persists for a set time period.

13. The system of claim 3, wherein the controller is configured to generate daily or weekly reports of oxygen saturation, activity level, and oxygen flow data accessible to a healthcare provider via a secure portal.

14. The system of claim 3, wherein communication between the wearable sensor device, the controller, and the smart flow regulator is achieved wirelessly using at least one of Bluetooth®, Wi-Fi, or cellular communication protocols.

15. The system of claim 3, wherein the controller is integrated in the smart flow regulator, the wearable sensor device, or an external device.

16. The system of claim 15, wherein the external device comprises a smartphone or tablet executing a software application configured to:
receive oxygen saturation and activity data from the wearable sensor device;
transmit flow control signals to the smart flow regulator;
display real-time physiological and system information; and
store data locally or on a remote server for review by healthcare providers.

17. The system of claim 1, wherein the wearable sensor device is further configured to monitor additional physiological parameters including heart rate, respiratory rate, skin temperature, and blood pressure, and wherein the controller adjusts oxygen delivery based on one or more of said parameters.

18. A method for automating oxygen monitoring and dosing in real time for a patient on oxygen therapy, comprising:
measuring oxygen saturation levels of the patient with a wearable sensor device;
tracking physical activity of the patient with the wearable sensor device;
transmitting real-time oxygen saturation and activity data from the wearable sensor device to a controller;
processing the transmitted data using an adaptive control algorithm executed by the controller to determine an oxygen flow rate appropriate for the patient's physiological and activity state; and
automatically adjusting a smart flow regulator operably coupled to an oxygen source to deliver oxygen at the determined flow rate.

19. The method of claim 18, further comprising classifying the activity level of the patient as resting or active based on actigraphy data, and applying corresponding baseline oxygen flow rates for each state.

20. The method of claim 18, wherein the adaptive control algorithm comprises a proportional-integral-derivative (PID) feedback system configured to incrementally adjust oxygen flow based on averaged oxygen saturation readings over a time window of 10-20 seconds.

21. The method of claim 18, further comprising maintaining the oxygen saturation level within a target range of 90% to 95% by:
increasing the flow rate when oxygen saturation falls below the target range; and
decreasing the flow rate when oxygen saturation exceeds the target range.

22. The method of claim 18, further comprising triggering an alarm and notifying the patient to rest or seek assistance when the oxygen saturation level falls below a critical threshold for a predetermined time period.

23. The method of claim 18, further comprising storing oxygen saturation, activity, and flow rate data locally or transmitting the data to a cloud database accessible to a healthcare provider.

24. The method of claim 18, wherein the smart flow regulator comprises a stepper motor operably connected to a mechanical or digital flow control member of an oxygen concentrator or tank.

25. The method of claim 18, further comprising averaging oxygen saturation readings over a predetermined interval to account for physiologic delay in oxygen uptake prior to adjusting the flow rate.

26. The method of claim 18, further comprising automatically initiating an emergency call or provider alert if oxygen saturation remains below a critical level for a predetermined duration.

27. The method of claim 18, wherein communication between the wearable sensor device, the controller, and the smart flow regulator is performed via a wireless communication protocol selected from the group consisting of Bluetooth®, Wi-Fi, and cellular communication.

28. The method of claim 18, further comprising generating and displaying in a software application a graphical summary of oxygen saturation trends, activity levels, and corresponding oxygen flow rates for review by a healthcare provider.

29. The method of claim 18, wherein the adaptive control algorithm further utilizes historical patient data to predict oxygen needs and pre-emptively adjust the flow rate during anticipated periods of exertion.

30. A method for personalized oxygen delivery to a user on oxygen therapy, comprising:
continuously monitoring an oxygen saturation level of the user with a wearable oxygen sensor;
tracking, in real time, a physical activity level of the user using an activity sensor integrated with or communicatively coupled to the wearable oxygen sensor;
dynamically adjusting a flow rate of oxygen delivered from an oxygen source to the user in response to at least one of the detected physical activity level; and the measured oxygen saturation level;
incrementally increasing or decreasing the oxygen flow rate at predetermined intervals in accordance with an adaptive titration protocol, thereby maintaining the oxygen saturation level within a predetermined range; and
reassessing the oxygen saturation data after each incremental flow rate adjustment to determine whether further adjustment is required.

31. The method of claim 30, wherein the reassessment of oxygen saturation occurs approximately every two minutes following each flow rate adjustment.

32. The method of claim 30, wherein said incrementally adjusting the oxygen flow rate comprises increasing the flow rate by predetermined increments when the oxygen saturation falls below a threshold level and decreasing the flow rate when the oxygen saturation exceeds an upper limit.

33. The method of claim 30, further comprising classifying the user's activity level as one of a resting state or an active state, and initiating adjustment of the flow rate from respective baseline oxygen flow rates associated with the classified state.

34. The method of claim 30, wherein said incrementally adjusting the oxygen flow rate is performed automatically by a controller executing an adaptive algorithm configured to interpret both oxygen saturation and activity data.

35. The method of claim 30, wherein the adaptive algorithm comprises a proportional-integral-derivative (PID) feedback control that smooths variations in oxygen saturation and compensates for physiological delay in oxygen uptake.

36. The method of claim 30, further comprising averaging measured oxygen saturation values over a predetermined time window of about 10-20 seconds prior to each reassessment.

37. The method of claim 30, further comprising triggering an alert when the oxygen saturation falls below a predetermined critical threshold for longer than a preset duration.

38. The method of claim 30, wherein the incremental adjustment of the oxygen flow rate occurs in discrete steps of approximately 1 liter per minute (L/min) or less.

39. The method of claim 30, wherein the predetermined reassessment interval and adjustment increments are configurable by a healthcare provider via a software interface.

40. The method of claim 30, further comprising storing oxygen saturation, activity level, and flow rate data in a local or cloud-based memory for review by a healthcare provider.

41. The method of claim 30, wherein said incrementally adjusting the oxygen flow rate is based solely on detected physical activity when valid oxygen saturation data are unavailable or outside a confidence threshold.

42. The method of claim 30, wherein the reassessment interval and incremental flow changes are coordinated such that no flow adjustment is performed until a stable oxygen saturation value has been confirmed over a preceding assessment window.

43. The method of claim 30, further comprising predicting upcoming oxygen demands based on historical activity data of the user and preemptively adjusting the oxygen flow rate to maintain desired oxygen saturation levels.

44. The method of claim 30, further comprising displaying, on a graphical user interface, real-time oxygen saturation, activity level, and current flow rate of oxygen delivery.
